SEQUENCE LISTING (2 sequences)

SEQ ID NO: 1 - protein, 112 aa, Homo sapiens [sequence omitted]

SEQ ID NO: 2 - DNA, 336 bp, Homo sapiens
  DNA encoding wild-type human TGF-Beta 3
gctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagc

(19) United States
(12) Patent Application Publication
(10) Pub. No.: US 2011/0152189 A1
(43) Pub. Date: Jun. 23, 2011

(54) METHODS FOR THE INHIBITION OF SCARRING

(75) Inventors: Mark William James Ferguson, Manchester (GB); Sharon O'Kane, Manchester (GB); Tracey Mason, Manchester (GB); Mark Cooper, Manchester (GB); Nick Occleston, Manchester (GB); David Fairlamb, Manchester (GB); James Bush, Manchester (GB)

(73) Assignee: RENOVO LIMITED, Manchester (GB)

(21) Appl. No.: 13/003,755

(22) PCT Filed: Dec. 11, 2008
(86) PCT No.: PCT/GB2008/004072

§ 371 Date: Jan. 11, 2011

(30) Foreign Application Priority Data
Dec. 12, 2007 (GB) 0724203.5

(51) Int. Cl. A61K 38/18; A61P 17/02

(52) U.S. Cl. 514/8.9

(57) ABSTRACT

The invention provides new methods of treatment using TGF-β3 to inhibit scarring in humans, and TGF-β3 for new uses in the inhibition of scarring in humans. In a first incidence of treatment each centimetre of wound margin, or each centimetre of a site at which a wound is to be formed, is provided with between approximately 350 ng and 1000 ng of TGF-β3; and in a second incidence of treatment, occurring after a wound is formed, and between 8 and 48 hours after the first incidence of treatment, the wound is provided with an amount of between approximately 350 ng and 1000 ng of TGF-β3 per centimetre of wound margin in which scarring is to be inhibited. The amount of TGF-β3 provided may be the same in each incidence of treatment. The amount of TGF-β3 provided per centimetre in each incidence of treatment may preferably be approximately 500 ng. The TGF-β3 may be provided by intradermal injection. Also provided are kits and methods of selecting an appropriate treatment regime for inhibiting scarring associated with the healing of a human wound.

The present invention relates to the provision of new methods for inhibiting scarring formed on healing of human wounds. The invention also provides new uses of TGF-β3; new methods of selecting an appropriate treatment regime for inhibiting scarring associated with the healing of a wound; and kits for use in the inhibition of scarring associated with healing of a wound.

The Transforming Growth Factor-Betas (TGF-βs) are a family of cytokines having diverse biological activities. The TGF-β family comprises five isoforms, TGF-β1, TGF-β2, TGF-β3, TGF-β4, and TGF-β5. The members of the TGF-β family naturally exist in the form of dimers comprising two peptide chains. Active TGF-β dimers have a molecular weight of approximately 25.4 kDa.

TGF-β3 has been shown to be useful in the prevention, reduction or inhibition of scarring at sites throughout the body. This effect is particularly advantageous in relation to TGF-β3's ability to inhibit scarring associated with the healing of wounds. The amino acid sequence of human TGF-β3 is shown in Sequence ID No. 1, and the sequence of a nucleic acid encoding TGF-β3 is shown in Sequence ID No. 2.

The scarring response to healing of a wound is common throughout all adult mammals. The scarring response is conserved between the majority of tissue types and in each case leads to the same result, formation of fibrotic tissue termed a “scar”. A scar may be defined as “fibrous connective tissue that forms at the site of injury or disease in any tissue of the body”.

In the case of a scar that results from healing of a wound, the scar constitutes the structure produced as a result of the reparative response. This reparative process has arisen as the evolutionary solution to the biological imperative to prevent the death of a wounded animal. In order to overcome the risk of mortality due to infection or blood loss, the body reacts rapidly to repair the damaged area, rather than attempt to regenerate the damaged tissue. Since the damaged tissue is not regenerated to attain the same tissue architecture present before wounding, a scar may be identified by virtue of its abnormal morphology as compared to unwounded tissue.

Viewed macroscopically, scars may be depressed below the surface of the surrounding tissue, or elevated above the surface of their undamaged surroundings. Scars may be relatively darker coloured than normal tissue (hyperpigmentation) or may have a paler colour (hypopigmentation) compared to their surroundings. In the case of scars of the skin, either hyperpigmented or hypopigmented scars constitute a readily apparent cosmetic defect. It is also known that scars of the skin may be redder than unwounded skin, causing them to be noticeable and cosmetically unacceptable. It has been shown that the cosmetic appearance of a scar is one of the major factors contributing to the psychological impact of scars upon the sufferer, and that these effects can remain long after the wound that caused the scar has healed.

In addition to their psychological effects, scars may also have deleterious physical effects upon the sufferer. These effects typically arise as a result of the mechanical differences between scars and normal tissue. The abnormal structure and composition of scars mean that they are typically less flexible than their normal tissue counterpart. As a result scars may be responsible for impairment of normal function (such as in the case of scars covering joints which may restrict the possible range of movement) and may retard normal growth if present from an early age.

In the light of the above it will be recognised that TGF-β3 is of great utility in the clinical management of scarring that may occur on healing of wounds, but that there also remains a requirement for new and improved methods of treatment that may be used to inhibit scarring associated with the healing of wounds.

It is an object of some aspects of the present invention to provide improved methods of inhibiting scarring formed on healing of wounds. It is an object of other aspects of the invention to provide new uses of TGF-β3. These new uses of TGF-β3 may constitute alternative uses to those known from the prior art, but it may be preferred that they constitute improved uses compared to those already known. It is an object of certain aspects of the invention to provide; new methods of selecting an appropriate treatment regime for inhibiting scarring associated with the healing of a wound. It is an object of other aspects of the invention to provide kits for use in the inhibition of scarring associated with healing of a wound. These kits may be used in methods of treatment that provide increased inhibition of scarring compared to those known from the prior art.

In a first aspect of the invention there is provided a method of inhibiting scarring formed on healing of a wound of a human, the method comprising treating a body site in which scarring is to be inhibited:

in a first incidence of treatment providing to each centimetre of wound margin, or each centimetre of a site at which a wound is to be formed an amount of between approximately 350 ng and 1000 ng of TGF-β3; and

in a second incidence of treatment, occurring after a wound is formed and between 8 and 48 hours after the first incidence of treatment, providing to said wound an amount of between approximately 350 ng and 1000 ng of TGF-β3 per centimetre of wound margin in which scarring is to be inhibited.

In a second aspect, the invention provides a method of inhibiting scarring formed on healing of a wound of a human, the method comprising treating a body site in which scarring is to be inhibited:

in a first incidence of treatment providing to each centimetre of a site where a wound is to be formed an amount of between approximately 350 ng and 1000 ng of TGF-β3; and

in a second incidence of treatment, occurring between 8 and 48 hours after the first incidence of treatment, providing to said wound an amount of between approximately 350 ng and 1000 ng of TGF-β3 per centimetre of wound margin in which scarring is to be inhibited.

In a third aspect, the invention provides a method of inhibiting scarring formed on healing of a wound of a human, the method comprising treating a body site in which scarring is to be inhibited:

in a first incidence of treatment providing to each centimetre of wound margin, or each centimetre of future wound margin, an amount of between approximately 350 ng and 1000 ng of TGF-β3; and

in a second incidence of treatment, occurring between 8 and 48 hours after the first incidence of treatment, providing to said wound an amount of between approximately 350 ng and 1000 ng of TGF-β3 per centimetre of wound margin in which scarring is to be inhibited.

In various aspects and embodiments of the invention, the present disclosure defines the amount of TGF-β3 to be provided to a body site with reference to the amount to be provided per centimetre of such a site (for example, per centimetre of a site to be wounded, or per centimetre of wound margin or of future wound margin). It will be appreciated that, while these passages define the amount of TGF-β3 to be provided to such sites, they do not limit the manner in which this amount is to be provided. In particular, these passages should not be taken as requiring the administration of TGF-β3 to each centimetre of a site to be treated (though this may be a preferred embodiment). The requisite TGF-β3 may be provided by any number of administrations occurring at any site that allows the specified amount of TGF-β3 to be provided to the site at which scarring is to be inhibited.

The present invention is based upon the inventors' finding that scarring that would otherwise be expected on healing of a human patient's wound can be surprisingly effectively inhibited by use of a two part treatment regime, in which the site where scarring is to be reduced is treated with between 350 ng and 1000 ng TGF-β3 at a time around wounding or wound closure, and then re-treated with a similar amount of TGF-β3 between 8 and 48 hours later. These treatment regimes, described for the first time in the present disclosure, give rise to scars that are much reduced compared to those obtainable using known methods of treatment.

Without wishing to be bound by any hypothesis, the inventors believe that exposure of the cells at a wound or a site where a wound will be formed to the TGF-β3 provided in the first incidence of treatment is able to reduce the scarring response (though, as described below, the doses considered in the present invention do not have the greatest anti-scarring effect if provided in a single incidence treatment) and the TGF-β3 provided in the second incidence of treatment may serve to counteract the pro-scarring ‘cascade’ of biological activities that otherwise arises at the wound site. This has a surprisingly beneficial effect in inhibiting scarring, which is noticeably greater than the effects that may be achieved using other TGF-β3 treatment regimes considered to date.

It will be appreciated that although the amount to be provided in each incidence of treatment is referred to in the present disclosure on the basis of the amount to be provided per centimetre, the disclosure is not limited by this, and may be used to determine suitable doses that may be applied to a wound measured by any suitable unit.

The finding underlying the invention is highly surprising since not only are the anti-scarring results achieved particularly effective, but the prior art would have lead the skilled person to believe that this treatment regime using two relatively high doses of TGF-β3 would not be of as much benefit as known regimes using smaller doses.

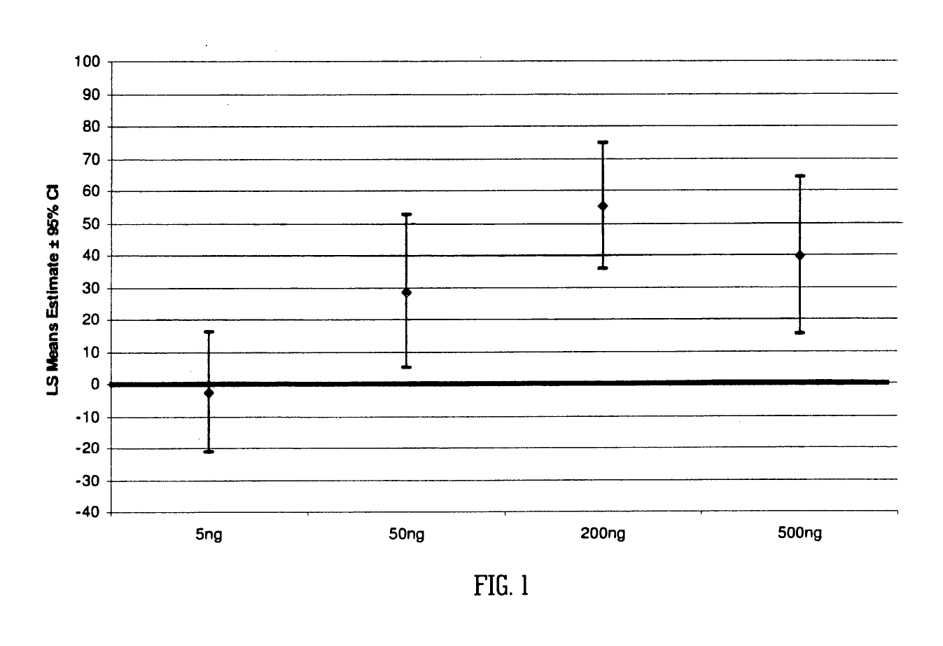

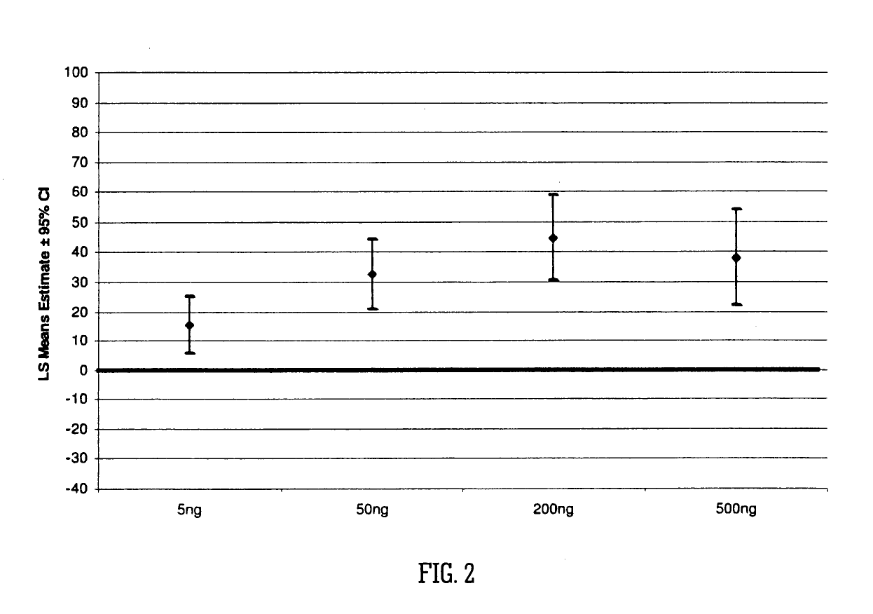

Previously it had been understood by those skilled in the art that the anti-scarring response to TGF-β3 took the form of a “bell shaped” dose response curve, of the sort shown in FIGS. 1 and 2. Doses at the upper or lower ends of this curve were not as effective as those positioned in the middle of the dose response, and data derived in animal models of wound healing had indicated that high doses of TGF-β3 may even increase collagen production in a way that would be expected to worsen scarring. Based on these findings a preferred therapeutically effective amount of TGF-β3 to be administered to a human subject per centimetre of a site in which scarring was to be inhibited had been identified as approximately 200 ng. Lower doses (of around 100 ng) or higher doses (such as 500 ng) did not give rise to such an effective reduction in scarring as did 200 ng. Investigations by the inventors, and by others working in this field, had determined that the 200 ng dose of TGF-β3 was effective to inhibit scarring of human patients when administered prior to wounding, or to the wound margins after a wound is formed.

Once studies into the anti-scarring effectiveness of TGF-β3 had identified an optimal dose to be used as being 200 ng, further investigations considered whether any advantage was conferred by repeated administration of this dose to a site where scarring was to be reduced. These results showed that repeated administration of 200 ng TGF-β3 to wounds did not provide any benefits in terms of the anti-scarring effect observed.

Given that single larger doses of TGF-β3, such as 500 ng, had been found to be less effective than smaller doses in achieving scar reduction, and that treatment regimes comprising multiple administrations of these preferred low doses had been shown not to provide any therapeutic advantage over single treatments, the skilled person would have been led to believe that regimes of the sort described in the first, second and third aspects of the invention would not be of any notable utility in the inhibition of scarring. These findings meant that the skilled person had no motivation to consider treatments of the sort described herein, in which relatively high doses are provided to the same site in repeated incidences of treatment. Indeed the skilled person would have considered that such treatments would likely be more costly and complicated, but less effective, than those already known to in the prior art. Thus it can be seen that the findings provided herein provide a surprising, but valuable, addition to the range of treatments that may be used to clinically inhibit the scarring of wounds.

The surprising advantages provided by the medicaments or methods of the invention, utilising repeated high doses of TGF-β3 had not previously been suggested either by human studies or experimental investigations carried out in non-human animals. Indeed there is nothing in the reports of previous human or animal studies that would even hint at the beneficial effects that the invention provides.

Recognition of the beneficial effects that may be achieved using the methods and medicaments of the invention also leads to a number of further aspects of the invention, which are also set out herein.

Since the methods of treatment disclosed herein require at least two incidences of treatment, which take place between at least 8 to 48 hours apart from one another, they are not suitable for use in patients that would not be able to complete a second, or further incidence of treatment. This observation gives rise to a further aspect of the invention, in which there is provided a method of selecting an appropriate treatment regime for inhibiting scarring associated with the healing of a wound, the method comprising:
determining whether an individual in need of such inhibition of scarring will be able to complete a second incidence of treatment occurring between 8 and 48 hours after a first incidence of treatment; and
if the individual will be able to complete a second incidence of treatment occurring between 8 and 48 hours after a first incidence of treatment, selecting a treatment regime comprising a method of treatment in accordance with any of the first three aspects of the invention, or
if the individual will not be able to complete a second incidence of treatment occurring between 8 and 48 hours after a first incidence of treatment, selecting a treatment regime comprising:
in a single incidence of treatment providing to each centimetre of wound margin, or each centimetre of a site at which a wound is to be formed, in which scarring is to be inhibited an amount of between approximately 150 and 349 ngTGF-β3. The amount of TGF-β3 provided per centimetre in this single incidence of treatment may preferably be approximately 200 ng.

In a further aspect of the invention there is provided the use of TGF-β3 for use as a medicament in treating a wound or site where a wound is to be formed to inhibit scarring, wherein in a first incidence of treatment the medicament is provided such that between approximately 350 ng and 1000 ng of TGF-β3 is provided to each centimetre of a wound margin or each centimetre of a site at which a wound is to be formed and wherein in a subsequent incidences of treatment the medicament is provided such that between approximately 350 ng and 1000 ng of TGF-β3 is provided to each centimetre of a wound margin between 8 hours and 48 hours after the previous incidence of treatment.

A medicament in accordance with this aspect of the invention may be a re-constitutable medicament, such as a lyophilised injectable composition.

Furthermore, the inventors have found that the means for effecting the methods of the invention, including medicaments manufactured in accordance with the invention, may usefully be provided in the form of a kit for use in the inhibition of scarring associated with healing of a wound, the kit comprising at least first and second vials comprising TGF-β3 for administration to a wound, or a site where a wound is to be formed, at times between 8 and 48 hours apart from one another.

In a further aspect of the invention there is provided a kit for use in the inhibition of scarring associated with healing of a wound, the kit comprising:

a first amount of a composition containing TGF-β3, this first amount being for administration to a wound, or a site where a wound is to be formed, in a first incidence of treatment;

a second amount of a composition containing TGF-β3, this second amount being for administration to a wound in a second incidence of treatment;

instructions regarding administration of the first and second amounts of the composition at times between 8 and 48 hours apart from one another;

wherein the composition is formulated to provide an amount of between approximately 350 ng and 1000 ng TGF-β3 to each centimetre of a tissue or organ to which it is administered.

It may be preferred that the composition contains TGF-β3 at a concentration of about 500 ng/100 μl. Additional concentrations useful in such a kit include any concentrations ranging from about 350 ng/100 μl to about 1000 ng/100 μl.

A composition provided in such a kit may be provided in a form suitable for reconstitution prior to use (such as a lyophilised injectable composition).

In a further embodiment of the invention TGF-β3 is provided by a subcutaneous implant or depot medicament system for the pulsatile delivery of TGF-β3 to a wound or site where a wound is to be formed to inhibit scarring, wherein in a first incidence of treatment the medicament is provided such that between approximately 350 ng and 1000 ng of TGF-β3 is provided to each centimetre of a wound margin or each centimetre of a site at which a wound is to be formed and wherein in a subsequent incidences of treatment the medicament is provided such that between approximately 350 ng and 1000 ng of TGF-β3 is provided to each centimetre of a wound margin between 8 hours and 48 hours after the previous incidence of treatment.

A medicament in accordance with this aspect of the invention may be formulated in for example a bulk-eroding system such as polylactic acid and glycolic acid (PLGA) copolymer based microspheres or microcapsules systems containing the TGF-β3 or blends of PLGA:ethylcellulose systems may also be used. A further medicament in accordance with this aspect of the invention may be formulated in a surface-eroding system wherein the TGF-β3 is embedded in an erodible matrix such as the poly(ortho) ester and polyanhydride matrices wherein the hydrolysis of the polymer is rapid. A medicament in accordance with this aspect of the invention may also be formulated by combining a pulsatile delivery system as described above and an immediate release system such as a lyophilised injectable composition described above. It will be appreciated that, while TGF-β3 may be administered by the same route and in the same form in each incidence of treatment, different incidences of treatment may provide TGF-β3 by different medicaments and/or different routes of administration. In preferred embodiments of the invention the initial incidence of treatment may provide TGF-β3 by means of an injection, such as an intradermal injection, while the second (and any subsequent) incidences of treatment may involve provision of TGF-β3 by alternative routes, such as topical formulations.

It may generally be preferred that the TGF-β3 used in the medicaments and methods of the invention is wild-type homodimeric TGF-β3, comprising two dimers each comprising the amino acid residue sequence illustrated in Sequence ID No. 1. The Experimental Results reported elsewhere in the present specification have been generated using this form of TGF-β3.

The inventors believe that the benefits that may be derived from the present invention may be applicable to wounds at sites throughout the body. However, it may be preferred that the wound, scarring associated with which is to be inhibited, is a skin wound. For illustrative purposes the embodiments of the invention will generally be described with reference to skin wounds, although they remain applicable to other tissues and organs. Merely by way of example, in another preferred embodiment the wound may be a wound of the circulatory system, particularly of a blood vessel (in which case the treatments may inhibit restenosis). Other wounds in which scarring may be inhibited in accordance with the present invention are considered elsewhere in the specification. The wound may be a result of surgery (such as elective surgery), and this constitutes a preferred embodiment of the invention.

The methods of the invention may optionally comprise a third or further incidence of treatment. Such further incidences of treatment may be continued as necessary until a clinician responsible for the care of the patient determines that a desired inhibition of scarring has been achieved. Each incidence of treatment should occur between 8 and 48 hours after the preceding incidence of treatment. Further guidance as to timing of third or further incidences of treatment may be taken from the disclosure herein relating to the relative timing of the first and second incidences.

It may be preferred that the first incidence of treatment occurs prior to wounding, in which case TGF-β3 may be provided to a site where a wound is to be formed. In the case that the TGF-β3 is provided by local injection to the skin (such as intradermal injection) this may cause a bleb to be raised as a result of the introduction of a TGF-β3 containing solution into the skin. In one preferred embodiment the bleb may be raised in the site where the wound is to be formed, and indeed the wound may be formed by incising the bleb. In this case the amount of TGF-β3 to be provided in the first incidence of treatment may be determined with reference to the length of the site where the wound is to be formed.

Alternatively two blebs may be raised, on either side of the site where the wound is to be formed. These blebs may preferably be positioned within half a centimetre of where the margins of the wound will be formed. In this case the amount of TGF-β3 to be provided in the first incidence of treatment may be determined with reference to the length of the wound to be formed, measured in centimetres of future wound margin (defined below).

Preferably a bleb used to provide TGF-β3 to a site prior to wounding may cover substantially the full length of the site where the wound is to be formed. More preferably the bleb may extend beyond the length of the site where a wound is to be formed. Suitably such a bleb may extend around half a centimetre (or more) beyond each end of the wound to be formed.

Intradermal injections in accordance with these embodiments of the invention may be administered by means of a hypodermic needle inserted substantially parallel to the midline of the wound to be formed, or parallel to the margins of the wound to be formed. Injection sites may be spaced approximately one centimetre apart from one another along the length of the region to which TGF-β3 will be provided.

In the alternative, it may be preferred that the first incidence of treatment involves provision of TGF-β3 to an existing wound. The inventors believe that the biological mechanisms relevant to the anti-scarring activity are the same whether cells are exposed to TGF-β3 before or after wounding. In either case, the necessary biological activity may be achieved as long as the cells at the site where scarring is to be inhibited are exposed to a therapeutically effective amount of between approximately 350 and 1000 ng TGF-β3 either before or after wounding.

In embodiments of the invention in which TGF-β3 is to be provided to an existing wound, the requisite amount may be determined with reference to the length of the wound, measured in centimetres of wound margin (as discussed below). TGF-β3 should preferably be provided along the entire length of each wound margin, and may even be provided beyond the wounded area. In a preferred embodiment TGF-β3 may be provided along a length extending about half a centimetre (or more) beyond the ends of the margins of the wound.

Intradermal injection also represents a preferred route by which TGF-β3 may be provided to an existing wound. Intradermal injections provided in accordance with this embodiment should be administered to each margin of the wound. The site of injection may preferably be within half a centimetre of the edge of the wound. The injections may be administered by means of a hypodermic needle inserted substantially parallel to the edge of the wound. Injection sites may be spaced approximately one centimetre apart from one another along the length of the region to be treated.

The considerations set out in the preceding paragraphs in relation to provision of TGF-β3 to a wound in the first incident of treatment will also be applicable to its provision in second (or further) incidents. Since the second incidence of treatment takes place after wounding has occurred this will always involve provision of TGF-β3 to an existing wound. The wound may be open or closed, depending on the wound management strategy that is being applied.

When the first incidence of treatment involves provision of TGF-β3 to a site where a wound is to be formed it may be preferred that this provision occurs an hour or less before wounding is initiated, preferably half an hour or less before wounding is initiated, still more preferably a quarter of an hour or less before wounding is initiated, and most preferably ten minutes or less before wounding is initiated.

If the first incidence of treatment is to involve provision of TGF-β3 to an existing wound, the time at which this treatment is provided may be selected with reference to time elapsed after the wound has been formed. In this case, it may be preferred that a first incidence of treatment in accordance with the invention is initiated within two hours of wounding, preferably within one and a half hours of wounding, more preferably within an hour of wounding, still more preferably within half an hour of wounding, and most preferably within a quarter of an hour of wounding.

Alternatively or additionally, the timing of the first incidence of treatment may be selected with reference to the time elapsed after closure of the wound to be treated. In this case, it may be preferred that a first incidence of treatment in accordance with the invention is initiated within two hours of the closure of the wound being completed, preferably within one and a half hours of closure of the wound being completed, more preferably within an hour of closure of the wound being completed, still more preferably within half an hour of closure of the wound being completed, and most preferably within a quarter of an hour of closure of the wound being completed. In the case that a wound is not to be completely closed for clinical reasons (for example if it is necessary to maintain access to a site within the wound) closure of the wound may still be considered to have been completed once the wound is closed to the fullest extent that will be closed as part of the procedure undertaken.

It will be appreciated that selection of the timing of the first incidence of treatment with reference to the time elapsed after closure of the wound may be of particular relevance in the case of protracted surgical procedures, where a wound must be kept open for a prolonged time in order to allow access to a site where surgery is being performed.

The time elapsing between incidences of treatment will be between 8 and 48 hours. More preferably the time elapsing should be at least, more preferably at least 10 hours, even more preferably at least 12 hours, yet more preferably at least 14 hours, still more preferably at least 16 hours, yet more preferably still at least 18 hours, more preferably still 20 at least hours, ever more preferably at least 22 hours, and most preferably is approximately 24 hours.

The time elapsing between incidences of treatment may be up to 48 hours, but will preferably be up to approximately 44 hours, more preferably up to approximately 40 hours, even more preferably up to approximately 36 hours, yet more preferably up to approximately 32 hours, still more preferably up to approximately 28 hours, and most preferably is approximately 24 hours.

The amount of TGF-β3 to be provided per centimetre of a region to be treated (be it a site where a wound is to be formed, a future wound margin or the margin of an existing wound) is between approximately 350 ng and approximately 1000 ng. The amount to be provided may preferably be approximately 350 ng, 400 ng, or 450 ng. The amount to be provided may preferably be more than 350 ng, more preferably more than 400 ng, and even more preferably may be more than 450 ng. The amount to be provided may preferably be approximately 1000 ng, 900 ng, 800 ng, 700 ng or 600 ng. The amount to be provided may preferably be less than 1000 ng, more preferably less than 900 ng, still more preferably less than 800 ng, even more preferably less than 700 ng and yet more preferably less than 600 ng. Upper and lower limits of the amount to be provided may be selected independently from those listed in the preceding sentences. Most preferably the amount of TGF-β3 to be provided is approximately 500 ng.

Whatever the amount of TGF-β3 provided, it may be preferred that the amount provided per centimetre of body site at which scarring is to be inhibited is substantially the same in each incidence of treatment. In particular, it may be preferred that an amount of approximately 500 ng TGF-β3 per centimetre of the body site at which scarring is to be inhibited is provided in each incidence of treatment.

In practicing the methods of the invention, the cells of the area in which scarring is to be inhibited should be “bathed” in a pharmaceutically acceptable solution comprising between about 350 ng and 1000 ng TGF-β3. This will create a local environment in which the cells are exposed to sufficient TGF-β3 to prevent scarring. Details of the therapeutically effective amounts of TGF-β3 that may be used to achieve this aim are defined in the first, second and third aspects of the invention.

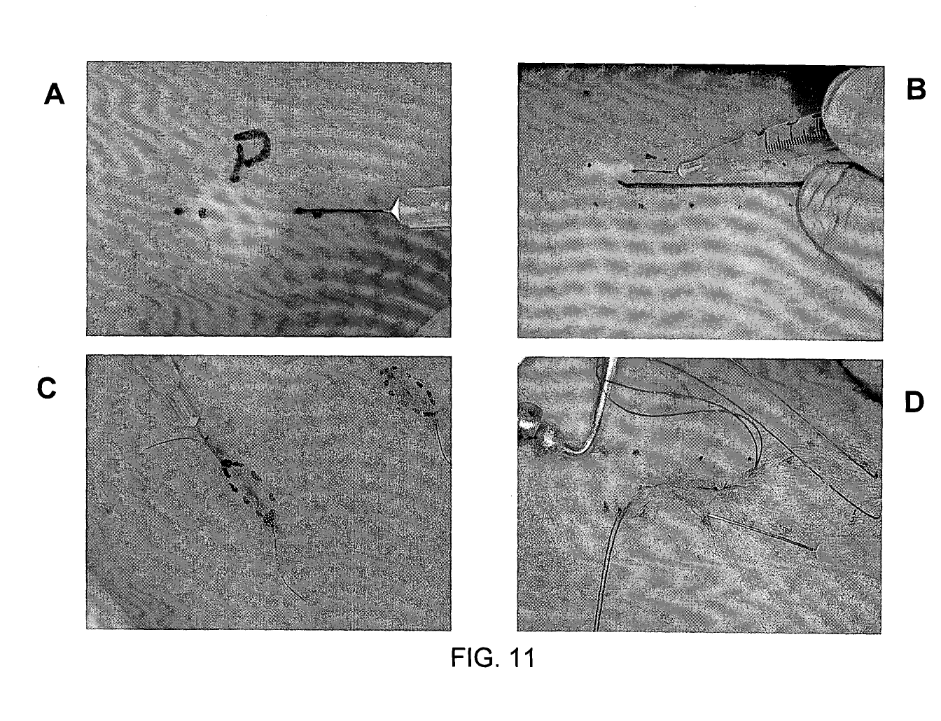

Cells that would otherwise be involved in scar formation will receive the therapeutically effective amount of TGF-β3 whether the TGF-β3 is administered by injection at the margins of a wound (or along the margins of a future wound—technique shown in panel B of FIG. 11), or by injection directly into the site at which the wound is to be formed (for example, by raising a bleb covering the site to be wounded—technique shown in panel A of FIG. 11). Either of these routes of administration are able to establish an anti-scarring concentration of TGF-β3 in the area surrounding the cells. However, it will be appreciated that the total amount of TGF-β3 that must be provided to the body site to achieve the required local environment for the cells will differ according to the route of administration used.

When the first incidence of treatment utilises injection directly into the site to be wounded, the requisite amount of TGF-β3 may be established around the cells by administration of a single injection (or series of “single” injections) administered along the line of the future wound and which cover the area to be wounded (technique illustrated in panel A of FIG. 11). When the first incidence of treatment utilises “paired” injections to each margin of a wound (or injections down each future margin of a wound—technique illustrated in panel B of FIG. 11) it will be appreciate that the total amount of TGF-β3 to be provided will be larger than that provided via the single injection route (described above), since injections on each margin are required in order to treat the same area. However, despite the differences in the total amount of TGF-β3 provided the biological effect on the target cells, and hence the therapeutic effect, does not differ between these two routes of administration.

It is preferred that TGF-β3 be provided to the requisite body site in the methods of the invention by means of an administration of a suitable pharmaceutical composition. Generally, any pharmaceutically acceptable solution may be used, but the inventors have found that compositions for use in accordance with the invention may advantageously comprise a sugar such as maltose or trehalose. Such sugars may serve to stabilise the composition, and also increase the biological activity of TGF-β3 so compounded. Preferred compositions may be those suitable for injection, and in particular for intradermal injection. Many formulations of compositions that may be used for the administration of TGF-β3 by intradermal injection will be known to those skilled in the art. Examples of suitable formulations are described in the inventors' co-pending patent application, published as WO 2007/007095, and formulations of the sort described in this application were used in the studies reported in the Experimental Results section of the present specification.

Various terms used in the present disclosure will now be described further for the avoidance of doubt. It will be appreciated that, for the sake of brevity, some of these terms may be described with reference to only certain aspects of the invention. However, except for where the context requires otherwise, the following descriptions of these terms will be applicable to all aspects of the invention.

Calculation of TGF-β3 Content, Potency and Amounts Administered

The protein content of solutions containing TGF-β3 (and in particular recombinant human TGF-β3, which is a preferred form of TGF-β3 to be used in accordance with the invention) may preferably be determined by quantitative Enzyme-linked Immunosorbent Assay (ELISA) calibrated with the United Kingdom National Institute for Biological Standards and Control (NIBSC) Transforming Growth Factor Beta-3 (Human rDNA derived) Reference Reagent code 98/608. Determination of protein content in this manner allows the concentration of solutions, and thus the amount of TGF-β3 that will be provided to a centimetre of a body site by a given volume of a solution, to be calculated by the skilled person. This protocol has been used in determining the protein content of solutions used in the Experimental Results section.

In the event that a skilled person is not able to obtain a reference sample of NIBSC Reference Reagent code 98/608, the inventors have found that ELISAs using their own TGF-β3 product (Lonza Bulk Drug Substance Lot 205-0505-005) as a standard give rise to values that are approximately 52% of those obtained with NIBSC Reference Reagent code 98/608. In the event that it is wished to use this alternative standard, instead of NIBSC Reference Reagent code 98/608, required amounts of TGF-β3 should be determined accordingly.

The biological activity (i.e. potency) of TGF-β3 to be used in accordance with the present invention may be determined by the inhibition of proliferation of Mink Lung Epithelial Cell line (MLEC); American Type Culture Collection (ATCC) Cat No. CCL-64. In a preferred embodiment, biological activity may be quantified by means of an assay calibrated using the United Kingdom National Institute for Biological Standards and Control Reference Reagent code 98/608, referred to above. Reference Reagent code 98/608 is considered to have a specific biological activity of 10 000 Arbitrary Units (AU) per microgram of TGF-β3 protein, and, by comparing the MLEC-inhibitory-activity of a sample of interest with the MLEC-inhibitory-activity of Reference Reagent code 98/608, the biological activity of the sample of interest in AU can be readily determined.

Thus a dose of 500 ng of TGF-β3 provides 5,000 AU of TGF-β3 activity. The inventors believe that a similar therapeutic effect may be achieved by an amount of TGF-β3 activity between approximately 3500 and 6500 AU. References to the use of 500 ng of TGF-β3 in the present disclosure may be construed accordingly.

Centimetre of a Site where a Wound is to be Formed

For ease of reference, the length of a site where a wound is to be formed may be measured in centimetres in order to determine the amount of TGF-β3 that will need to be provided in order to reduce scarring in accordance with the invention. It may be preferred that the length to be treated be calculated to extend beyond the intended length of the wound to be formed, in order to ensure that a therapeutically effective amount of TGF-β3 is provided to the ends of the wound. Accordingly, it may be preferred that the calculated length of a site where a wound is to be formed (and hence the length of the site to be treated) extend by a distance of about half a centimetre (or more) beyond each end of the intended wound.

Centimetre of Future Wound Margin

For the purposes of the present disclosure the length of a site where a wound is to be formed, as measured in number of centimetres of future wound margin, should be calculated as the sum of the lengths of each margin of the wound to be formed (in centimetres). It may be preferred that the length to be treated be calculated to extend beyond the ends of the margins of the wound to be formed, and this may help to ensure that a therapeutically effective amount of TGF-β3 is provided to the ends of the wound. Accordingly, it may be preferred that the calculated length of a future wound margin (and hence the length of the site to be treated) extend by a distance of about half a centimetre (or more) at each end of the wound to be formed.

Centimetre of Wound Margin

For the purposes of the present disclosure, the length of a wound, as measured in number of centimetres of wound margin, should be calculated as the sum of the lengths of each margin of the wound (in centimetres). It may be preferred that the length of the site to be treated be calculated to extend beyond the ends of the margins of the wound. This may help to ensure that a therapeutically effective amount of TGF-β3 is provided to the ends of the wound. Accordingly, it may be preferred that the calculated length of a wound margin to be treated in accordance with the invention extend by a distance of about half a centimetre (or more) beyond each end of the wound.

TGF-β3

For the purposes of the present disclosure, TGF-β3 may be taken to comprise a peptide comprising the amino acid sequence shown in Sequence ID No. 1. The TGF-β3 may preferably be dimeric TGF-β3, but the inventors believe that the inhibition of scarring described herein may also be achieved using monomeric forms of TGF-β3.

The inventors believe that the inhibition of scarring described in the present disclosure may also be achieved using therapeutically effective fragments or derivatives of TGF-β3. Fragments of TGF-β3 may readily be determined with reference to the sequence information provided in Sequence ID No. 1, and derivatives may be prepared based on this sequence information using means well known to those skilled in the art. Examples of suitable derivatives are disclosed in the inventors' co-pending application published as WO2007/104845.

The therapeutic effectiveness of such fragments or derivatives of TGF-β3 may be readily assessed with reference to any one of a number of suitable experimental models. Such models may include in vitro models indicative of biological effectiveness (which may be expected to correlate with therapeutic effectiveness), or in vivo studies using human or non-human subjects. Merely by way of example, the techniques described in the Experimental Results section set out elsewhere in the specification may be used or adapted in order to investigate therapeutic effectiveness of fragments or derivatives of TGF-β3.

In the event that it is desired to use forms of TGF-β3 other than the wild type dimeric active fragment (comprising two peptide chains corresponding to Sequence ID No.1), it will be appreciated that such agents may have molecular weights that are not the same as that of the naturally occurring form. Accordingly, the therapeutically effective amounts of such agents to be used in the medicaments or methods of the invention may be varied, to reflect the differences in molecular weights. Thus, if a form of TGF-β3 having half the molecular weight of the wild type dimeric active fragment is to be used, then a suitable therapeutically effective amount will be half those set out elsewhere in the specification.

Prevention/Inhibition/Reduction/Minimisation of Scarring

The inhibition of scarring within the context of the present invention should be understood to encompass any degree of prevention, reduction, minimisation or inhibition in scarring achieved on healing of a wound treated in accordance with a method of the invention (or a kit or medicament of the invention) as compared to the level of scarring occurring on healing of a control-treated or untreated wound. For the sake of brevity, the present specification will primarily refer to “inhibition” of scarring utilising TGF-β3, however, such references should be taken, except where the context requires otherwise, to also encompass the prevention, reduction or minimisation of scarring using TGF-β3.

Pharmaceutically Acceptable

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the US Federal or a state government or listed in the U.S. Pharmacopoeia or other generally recognized pharmacopeias for use in animals, and more particularly in humans.

Pharmaceutical Compositions and Administration

While it is possible to use a composition provided by the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent and/or carrier. The excipient, diluent and/or carrier must be “acceptable” in the sense of being compatible with the other ingredients of the formulation and not deleterious to the recipient thereof.

The compositions of the invention can be formulated for administration in any convenient way for use in human or veterinary medicine. The invention therefore includes within its scope pharmaceutical compositions comprising a product of the present invention that is adapted for use in human or veterinary medicine.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Wounds

The inventors believe that methods of treatment using TGF-β3 in accordance with the present invention may be used to beneficially inhibit scarring in all types of wounds.

Examples of specific wounds in which scarring may be inhibited using the medicaments and methods of the invention include, but are not limited to, those independently selected from the group consisting of: wounds of the skin; wounds of the eye (including the inhibition of scarring resulting from eye surgery such as LASIK surgery, LASEK surgery, PRK surgery, glaucoma filtration surgery, cataract surgery, or surgery in which the lens capsule may be subject to scarring) such as those giving rise to corneal cicatrisation; wounds subject to capsular contraction (which is common surrounding breast implants); wounds of blood vessels; wounds of the central and peripheral nervous system (where prevention, reduction or inhibition of scarring may enhance neuronal reconnection and/or neuronal function); wounds of tendons, ligaments or muscle; wounds of the oral cavity, including the lips and palate (for example, to inhibit scarring resulting from treatment of cleft lip or palate); wounds of the internal organs such as the liver, heart, brain, digestive tissues and reproductive tissues; wounds of body cavities such as the abdominal cavity, pelvic cavity and thoracic cavity (where inhibition of scarring may reduce the number of incidences of adhesion formation and/or the size of adhesions formed); and surgical wounds (in particular wounds associated with cosmetic procedures, such as scar revision). It is particularly preferred that the medicaments and methods of the invention be used to prevent, reduce or inhibit scarring associated with wounds of the skin.

Assessment of Scarring

The extent of scarring, and so any inhibition of scarring achieved, may be assessed by macroscopic clinical assessment of scars. This may be achieved by the direct assessment of scars upon a subject; or by the assessment of photographic images of scars; or of silicone moulds taken from scars, or positive plaster casts made from such moulds. For the purposes of the present disclosure a “treated scar” should be taken to comprise a scar produced on healing of a wound treated in accordance with the present invention.

Macroscopic characteristics of a scar which may be considered when assessing scarring include:
i) Colour of the scar. Scars may typically be hypopigmented or hyperpigmented with regard to the surrounding skin. Inhibition of scarring may be demonstrated when the pigmentation of a treated scar more closely approximates that of unscarred skin than does the pigmentation of an untreated scar. Scars may often be redder than the surrounding skin. In this case inhibition of scarring may be demonstrated when the redness of a treated scar fades earlier, or more completely, or to resemble more closely the appearance of the surrounding skin, compared to an untreated scar. Colour can readily be measured, for example by use of a spectrophotometer.
ii) Height of the scar. Scars may typically be either raised or depressed as compared to the surrounding skin. Inhibition of scarring may be demonstrated when the height of a treated scar more closely approximates that of unscarred skin (i.e. is neither raised nor depressed) than does the height of an untreated scar. Height of the scar can be measured directly on the patient (e.g. by means of profilometry), or indirectly, (e.g. by profilometry of moulds taken from a scar).
iii) Surface texture of the scar. Scars may have surfaces that are relatively smoother than the surrounding skin (giving rise to a scar with a “shiny” appearance) or that are rougher than the surrounding skin. Inhibition of scarring may be demonstrated when the surface texture of a treated scar more closely approximates that of unscarred skin than does the surface texture of an untreated scar. Surface texture can also be measured either directly on the patient (e.g. by means of profilometry), or indirectly (e.g. by profilometry of moulds taken from a scar).
iv) Stiffness of the scar. The abnormal composition and structure of scars means that they are normally stiffer than the undamaged skin surrounding the scar. In this case, inhibition of scarring may be demonstrated when the stiffness of a treated scar more closely approximates that of unscarred skin than does the stiffness of an untreated scar.

A treated scar will preferably exhibit inhibition of scarring as assessed with reference to at least one of the parameters for macroscopic assessment set out in the present specification. More preferably a treated scar may demonstrate inhibited scarring with reference to at least two of the parameters, even more preferably at least three of the parameters, and most preferably at least four of these parameters (for example, all four of the parameters set out above).

The height, length, width, surface area, depressed and raised volume, roughness/smoothness of scars can be measured directly upon the subject, for example by using an optical 3D measurement device. Scar measurements can be made either directly on the subject, or on moulds or casts representative of the scar (which may be formed by making a silicone mould replica impression of the scar and subsequently creating a plaster cast from the silicone moulds). All of these methods can be analysed using an optical 3D measurement device, or by image analysis of photographs of the scar. 3D optical measurements have a resolution in the micrometer range along all axes which guarantees a precise determination of all skin and scar parameters. The skilled person will also be aware of further non-invasive methods and devices that can be used to investigate suitable parameters, including calipers for manual measurements, ultrasound, 3D photography (for example using hardware and/or software available from Canfield Scientific, Inc.) and high resolution Magnetic Resonance Imaging.

Inhibition of scarring may be demonstrated by a reduction in the height, length, width, surface area, depressed or raised volume, roughness or smoothness or any combination thereof, of a treated scar as compared to an untreated scar.

One preferred method for the macroscopic assessment of scars is holistic assessment. This may be accomplished by means of assessment of macroscopic photographs by an expert panel or a lay panel, or clinically by means of a macroscopic assessment by a clinician or by patients themselves. Assessments may be captured by means of a VAS (visual analogue scale) or a categorical scale. Examples of suitable parameters for the assessment of scarring (and thereby of any reduction of scarring attained) are described below. Further examples of suitable parameters, and means by which assessment of such parameters may be captured, are described by Duncan et al. (2006), Beausang et al. (1998) and van Zuijlen et al. (2002).

Assessment Using Visual Analogue Scale (VAS) Scar Scores. Assessments of scars may be captured using a scarring-based VAS. A suitable VAS for use in the assessment of scars may be based upon the method described by Duncan et al. (2006) or by Beausang et al. (1998). This is typically a 10 cm line in which 0 cm is considered an imperceptible scar and 10 cm a very poor hypertrophic scar. Use of a VAS in this manner allows for easy capture and quantification of assessment of scarring. VAS scoring may be used for the macroscopic and/or microscopic assessment of scarring.

Merely by way of example, a suitable macroscopic assessment of scarring may be carried out using a VAS consisting of a 0-10 cm line representing a scale, from left to right, of 0 (corresponding to normal skin) to 10 (indicative of a bad scar). A mark may be made by an assessor on the 10 cm line based on an overall assessment of the scar. This may take into account parameters such as the height, width, contour and colour of the scar. The best scars (typically of small width, and having colour, height and contour like normal skin) may be scored towards the “normal skin” end of the scale (the left hand side of the VAS line) and bad scars (typically large width, raised profile and with uneven contours and whiter colour) may be scored towards the “bad scar” end of the scale (the right hand side of the VAS line). The marks may then be measured from the left hand side to provide the final value for the scar assessment in centimetres (to 1 decimal place).

An alternative assessment of scarring (whether macroscopic assessment or microscopic assessment), involving the comparison of two scars or two scar segments (such as one treated segment and another segment untreated, or control treated) to determine which one has a preferred appearance, may be carried out using a VAS comprising two 100 mm VAS lines intersected by a vertical line. In a VAS of this sort, the two VAS lines correspond to the two scars being compared, while the vertical line represents zero (indicating that there is no perceptible difference between the scars compared). The extremes of 100% (100 mm at the end of either VAS line) indicate one of the scars has become imperceptible in comparison to the surrounding skin.

A particularly preferred method of assessing the macroscopic appearance of scars in this manner is referred to as The Global Scar Comparison Scale (GSCS). This scale has been positively received by the European Medicines Agency (EMEA) and accepted as a preferred scale by which scars may be assessed and clinically relevant endpoints associated with the inhibition of scarring determined. In particular, it may be preferred to use a version of the GSCS based on clinical panel assessment, this being viewed by the EMEA as particularly relevant.

When comparing a pair of scars using a VAS of this sort, such as the GSCS, an assessor first determines which of the scars has the preferred appearance, or if there is no perceptible difference between the two. If there is no perceptible difference this is recorded by placing a mark at the zero vertical line. If there is a perceptible difference, the assessor uses the worse of the two scars as an anchor to determine the level of improvement found in the preferred scar, and then marks the score on the relevant section of the scale (i.e, setting a scale according to the comparison of scar appearance). The point marked represents the percentage improvement over the anchor scar.

The inventors have found that use of VAS measures of this sort in assessing the macroscopic or microscopic appearance of scars offers a number of advantages. Since these VAS are intuitive in nature they, 1) reduce the need for extensive training using reference images of different scar severities in different skin types, making this tool relatively simple to deploy in a large phase 3 trial; 2) reduce variability of the data: one assessment of each scar pair is performed as opposed to two independent assessments of drug and placebo scars; 3) incorporate the well-established principles of VAS (i.e., a continuous distribution of data) and the benefits of ranking in the same scale; and 4) allow easier communication of drug effect (percentage improvement) to clinicians and patients.

The present invention will now be further described with reference to the accompanying Experimental Results section and Figures, in which:

FIG. 1 compares the anti-scarring activity of different doses of TGF-β3 provided to human wounds in a single incidence of treatment.

FIG. 2 compares the anti-scarring activity of different doses of TGF-β3 provided to human wounds in two incidences of treatment administered within approximately one hour of one another.

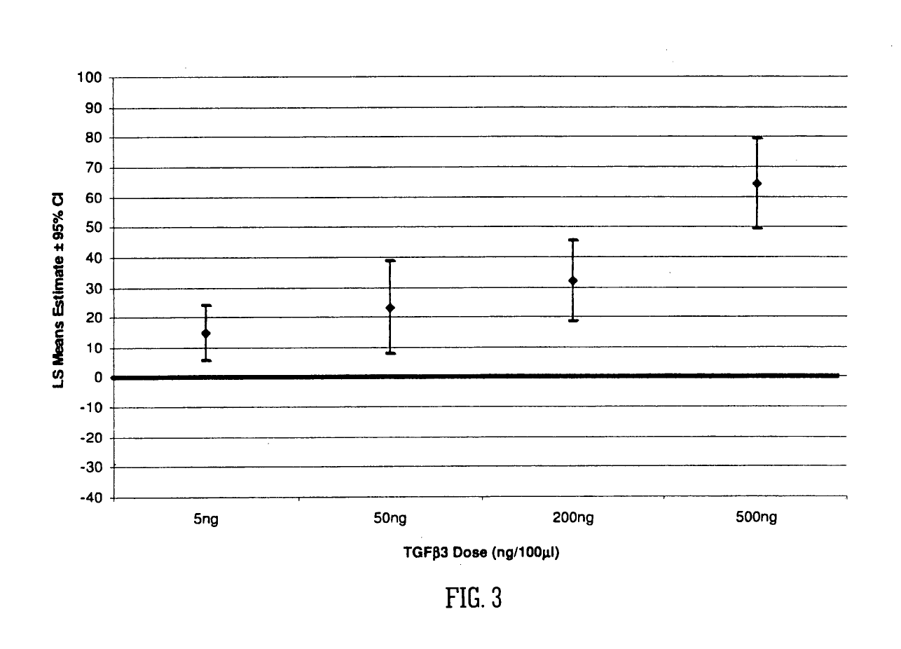

FIG. 3 compares the anti-scarring activity of different doses of TGF-β3 provided to human wounds in two incidences of treatment administered approximately 24 hours apart from one another. The treatment with two doses of 500 ng TGF-β3 is in accordance with the present invention, and can be seen to reduce scarring notably better than the other treatment regimes investigated.

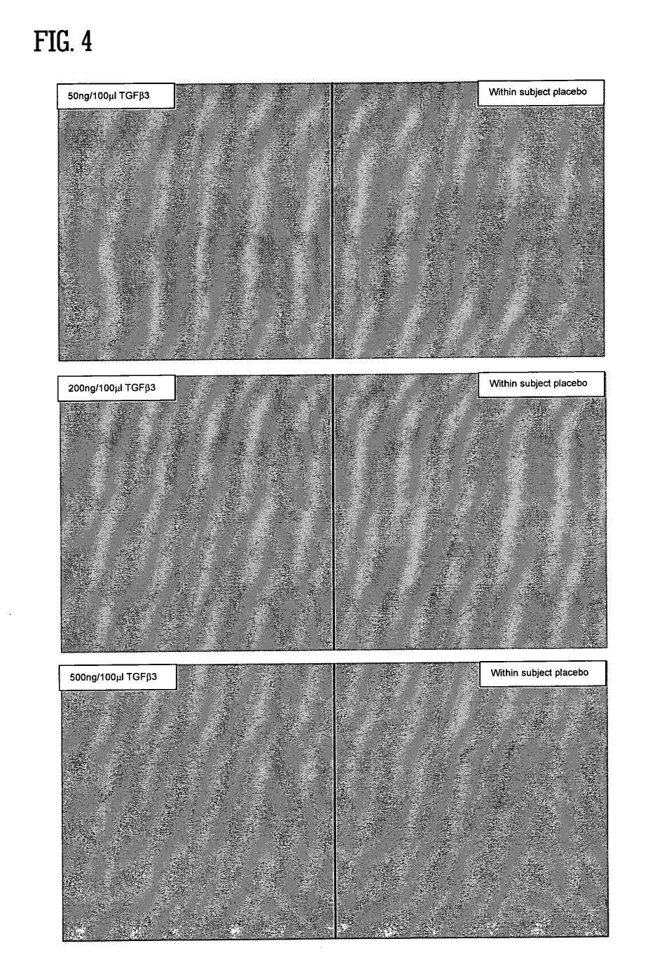

FIG. 4 compares macroscopic images of TGF-β3 treated scars or placebo treated control scars. The three TGF-β3-treated scars were provided with different amounts of TGF-β3 in incidences of treatment separated by about 24 hours.

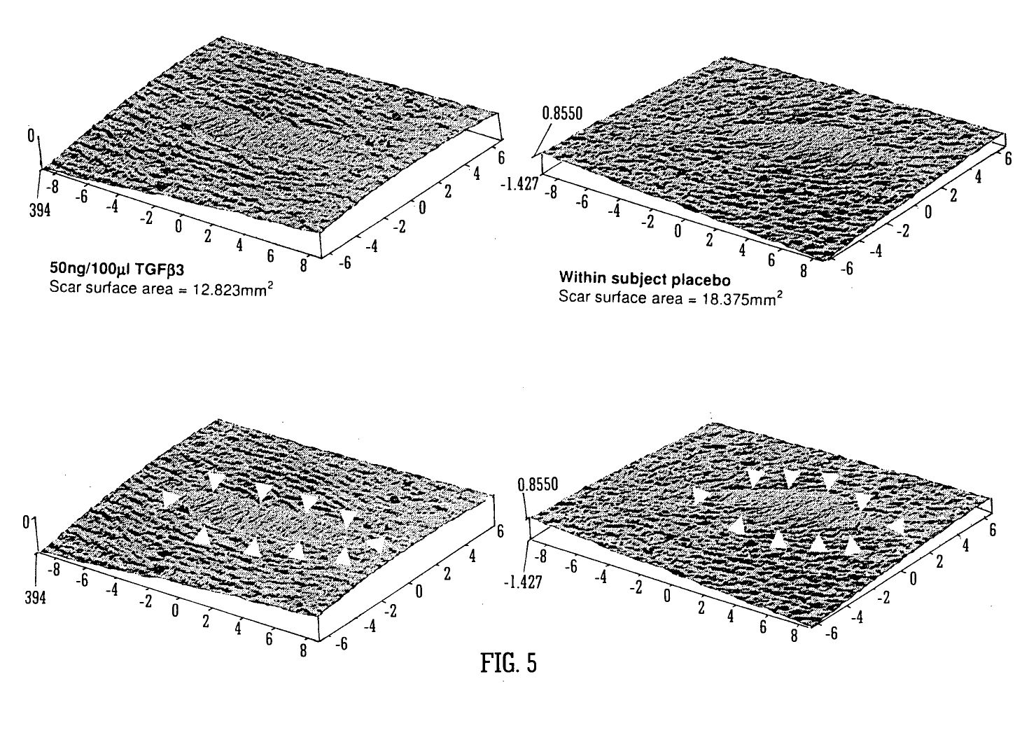

FIG. 5 illustrates 3-dimensional simulations and scar measurements taken from scars formed on healing of wounds treated with either TGF-β3 or placebo.

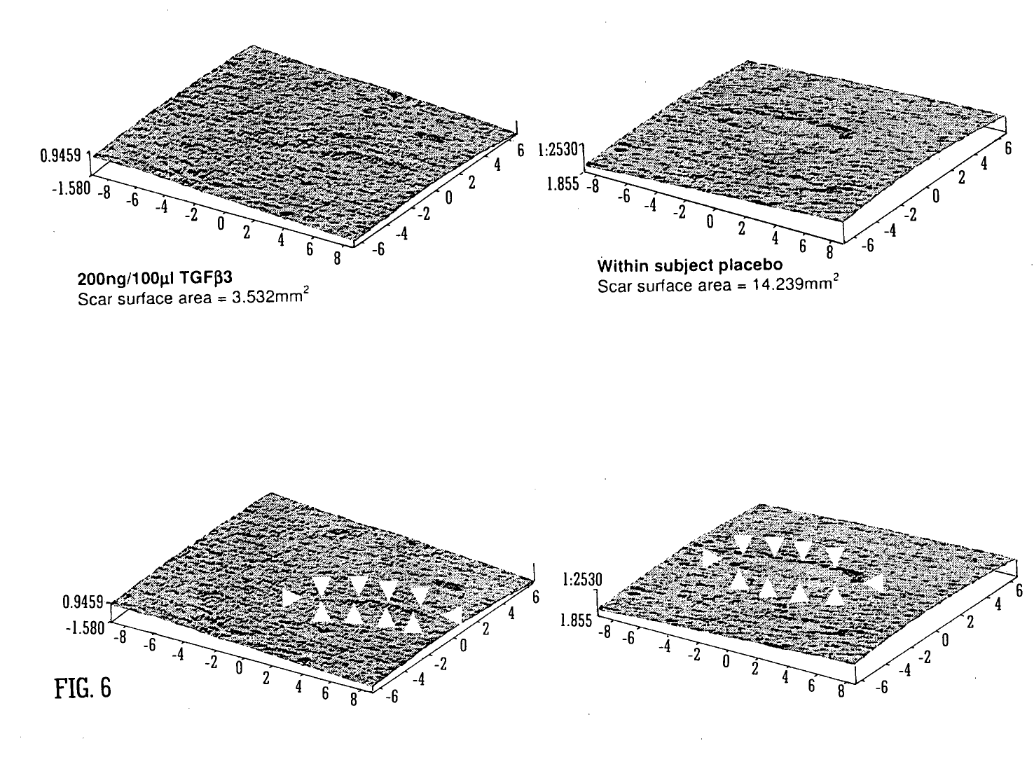

FIG. 6 illustrates 3-dimensional simulations and scar measurements taken from scars formed on healing of wounds treated with either TGF-β3 or placebo.

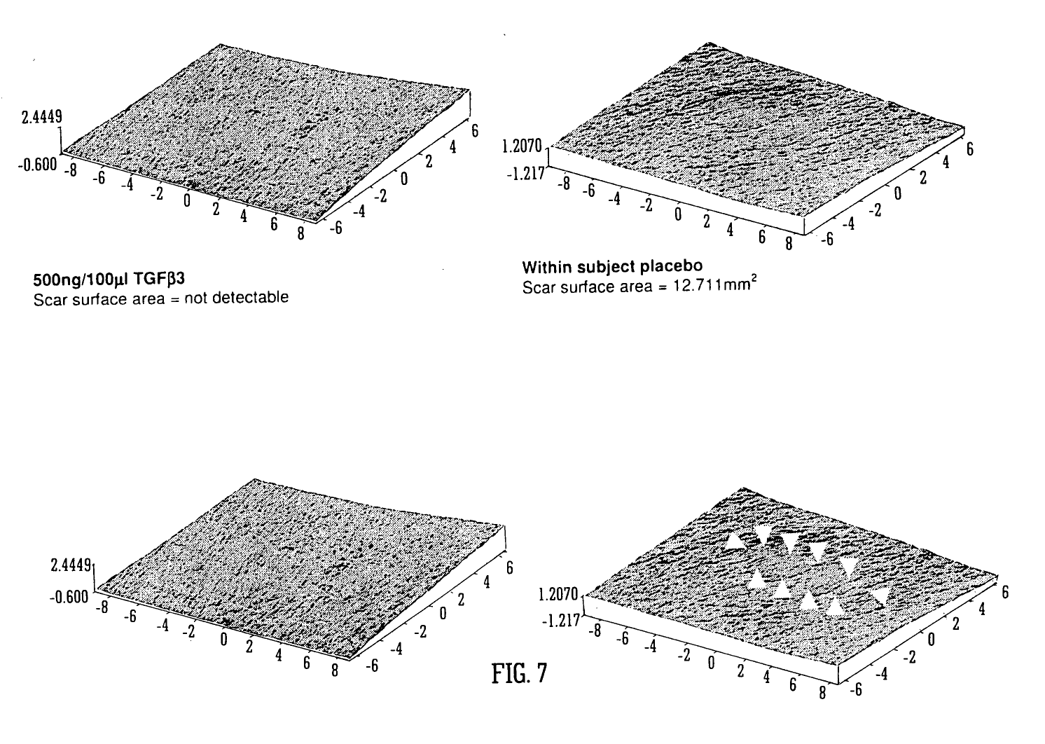

FIG. 7 illustrates 3-dimensional simulations and scar measurements taken from scars formed on healing of wounds treated with either TGF-β3 (in accordance with the present invention) or with placebo.

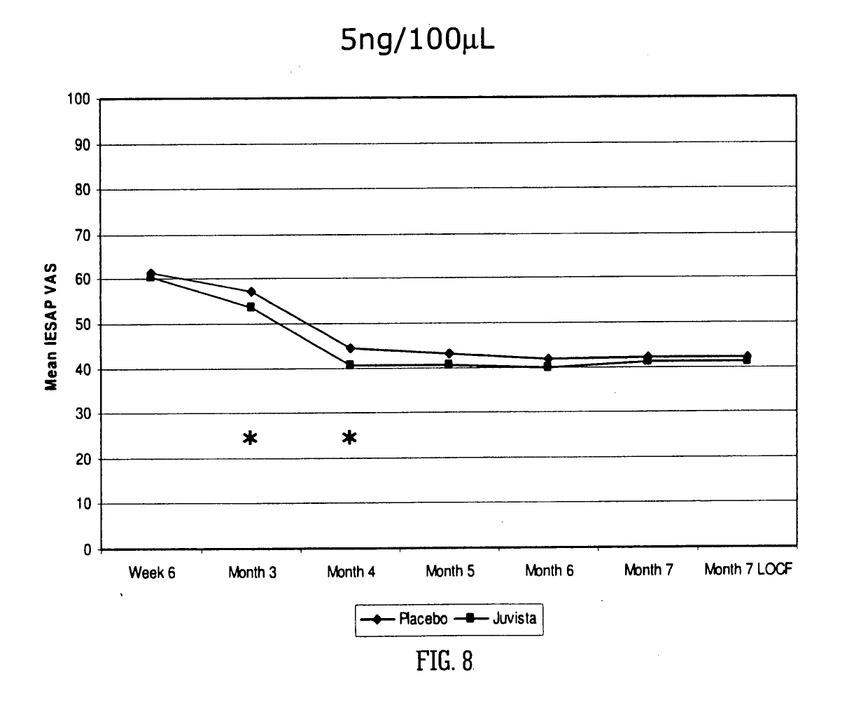

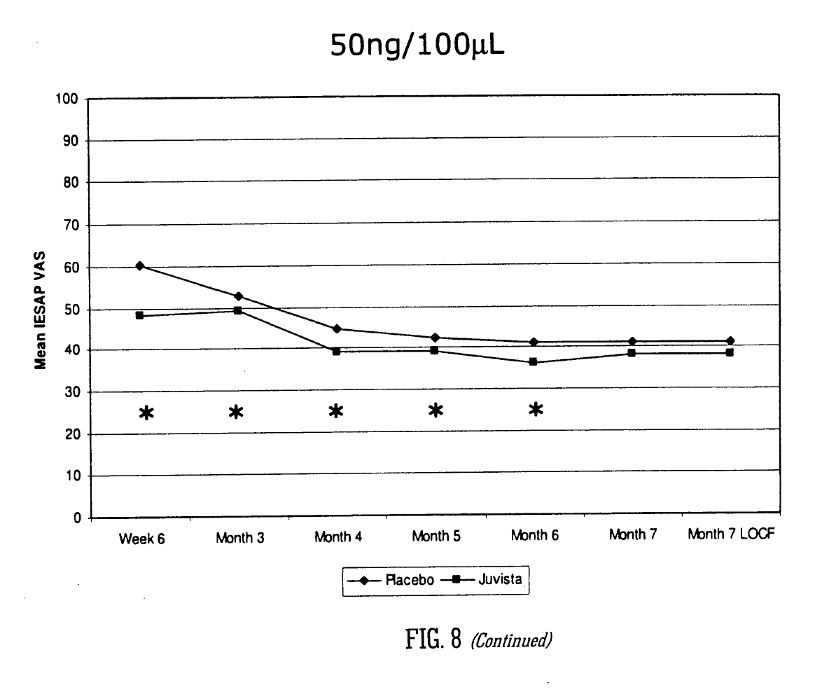

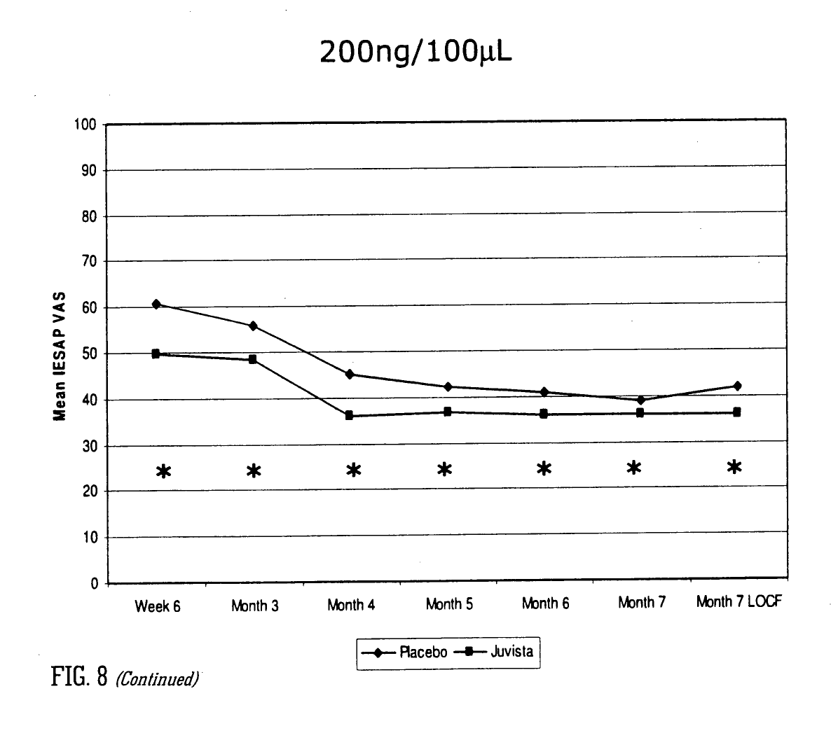

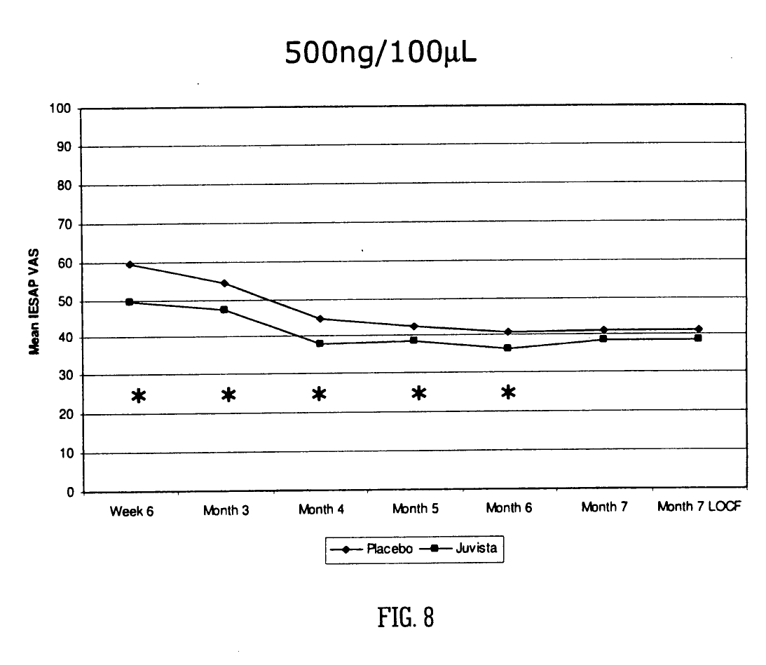

FIG. 8 compares the magnitude of inhibition of scarring achieved over time in scars formed on healing of wounds treated with one of four experimental regimes using TGF-β3 (administered in an amount of 5 ng, 50 ng, 200 ng or 500 ng per centimetre in each of two incidences of treatment separated by approximately one hour).

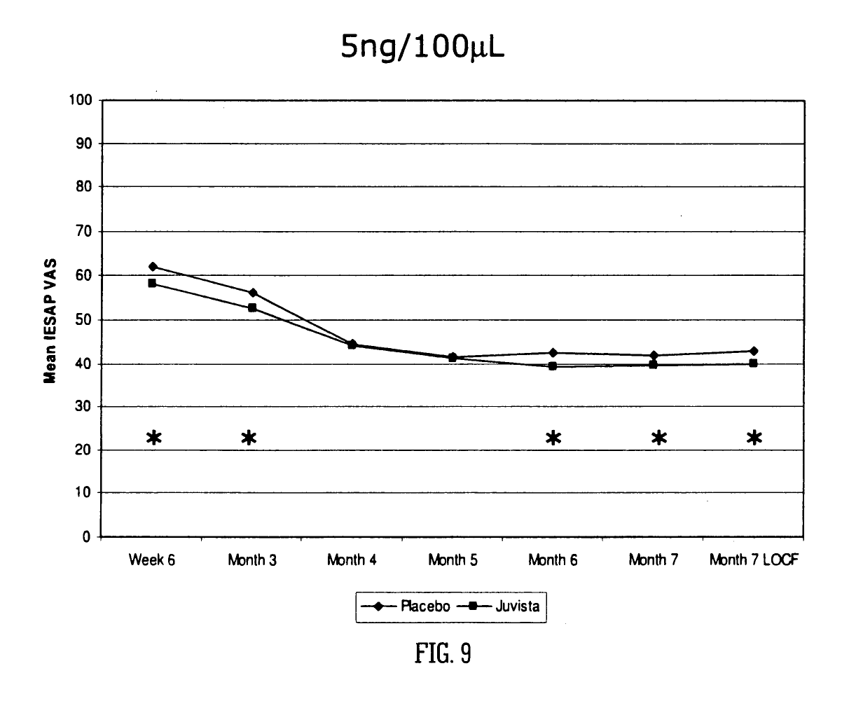

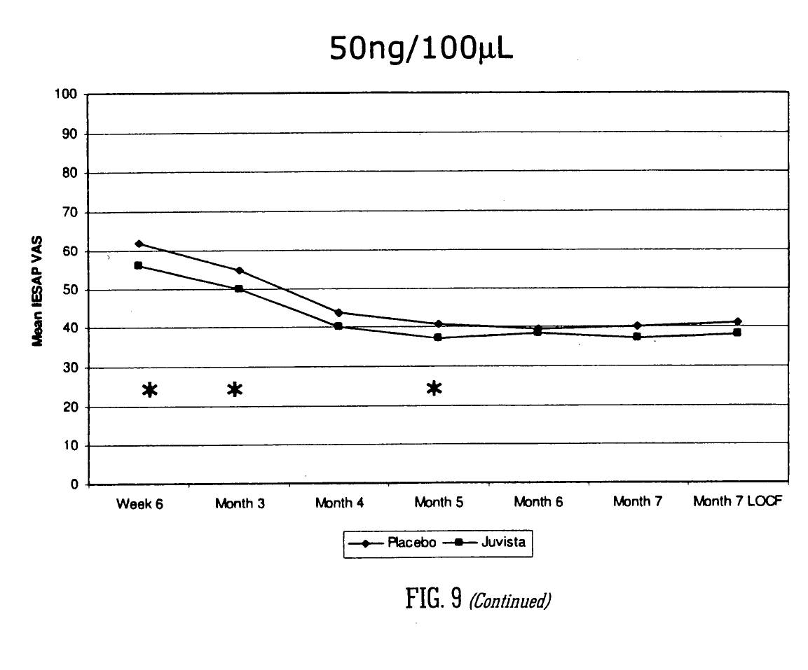

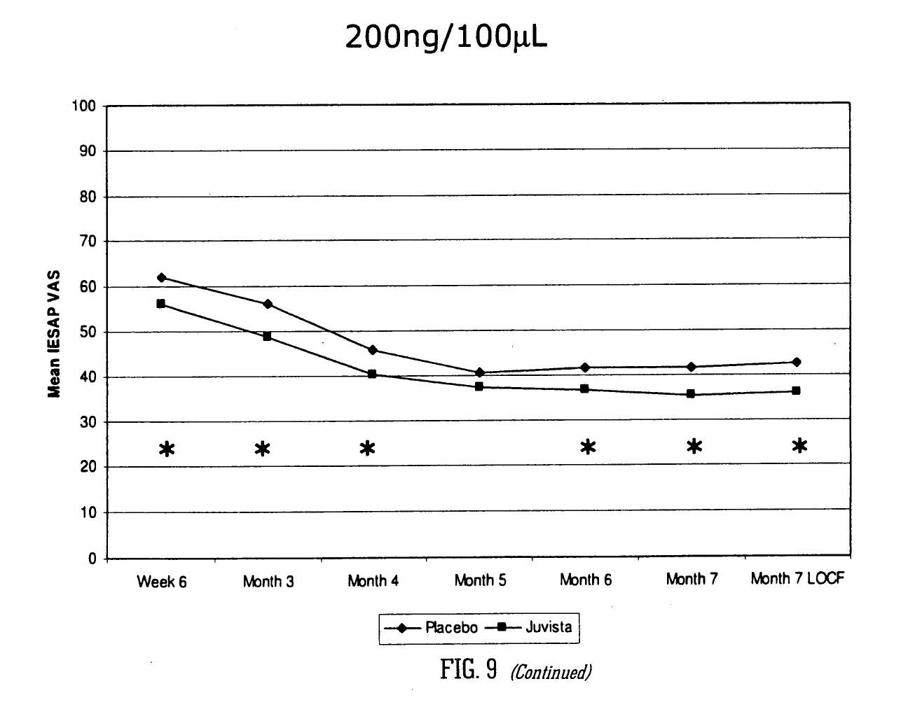

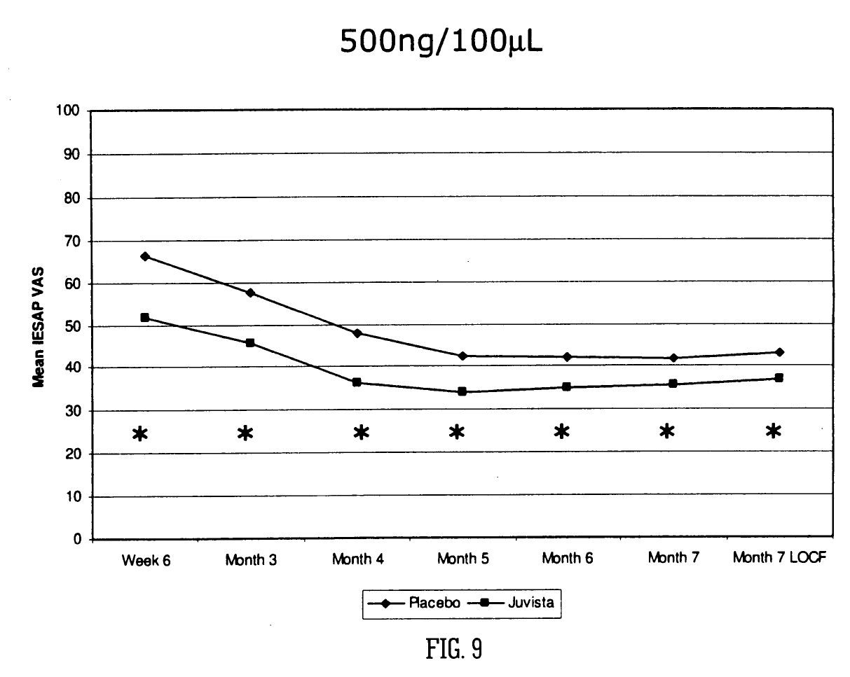

FIG. 9 compares the magnitude of inhibition of scarring achieved over time in scars formed on healing of wounds treated with one of four experimental regimes using TGF-β3 (administered in an amount of 5 ng, 50 ng, 200 ng or 500 ng per centimetre in each of two incidences of treatment separated by approximately 24 hours). The wounds treated with 500 ng have been treated in accordance with the present invention.

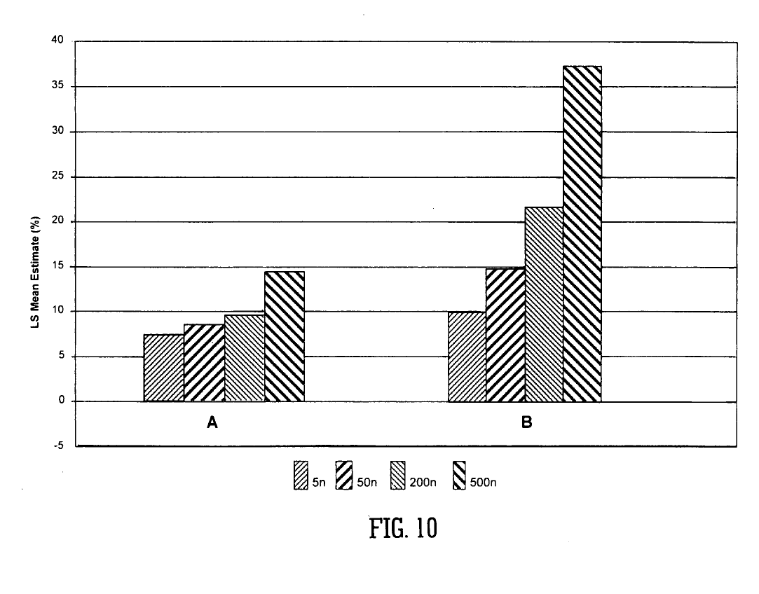

FIG. 10 illustrates results achieved using The Global Scar Comparison Scale to compare the magnitude of inhibition of scarring occurring in scars formed on healing of wounds treated with one of four experimental regimes using TGF-β3 (administered in an amount of 5 ng, 50 ng, 200 ng or 500 ng per centimetre in each of two incidences of treatment separated by approximately 24 hours). The wounds treated with 500 ng have been treated in accordance with the present invention. Assessment of scarring using the GSCS was made 12 months after wounding. The results marked as A are those achieved by GSCS assessment carried out on the patient by the investigating surgeon, while the results marked as B are those achieved by GSCS assessment carried out by an independent clinical expert panel.

FIG. 11 shows photographs illustrating preferred routes of administration that may be used to provide TGF-β3 to a body site at which it is wished to inhibit scarring in accordance with the present invention. Panel A shows administration of a single injection of a composition comprising TGF-β3 at a site to be wounded. This injection has raised a bleb that covers the site where the wound will be formed (between the two inner dots) and covers an area that extends beyond the intended wound site (the area bounded by the outer dots). Panel B shows the administration of a composition comprising TGF-β3 along a future wound margin. The solid line illustrates the site where a wound is to be formed, and sites at which TGF-β3 may be administered are shown by the dots that surround the future wound. Panels C and D illustrate administration of compositions comprising TGF-β3 to the margins of existing wounds (which have been closed with sutures).

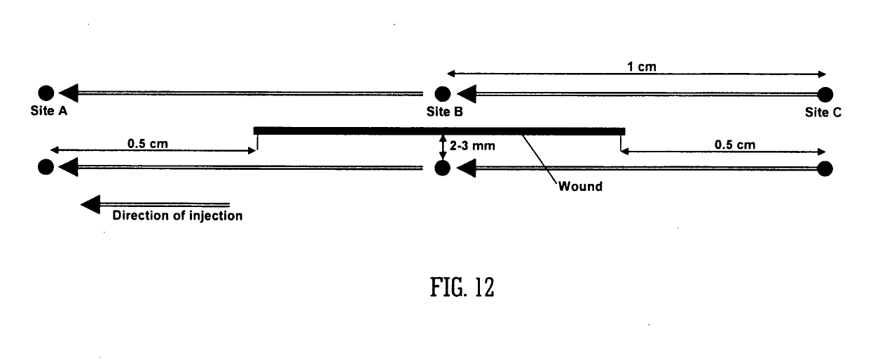

FIG. 12 illustrates a preferred method by which intradermal injections may be used for the administration of TGF-β3 in accordance with the present invention. A hypodermic needle through which TGF-β3 is to be administered is inserted intradermally at site B and advanced to site A (separated from site B by a distance of 1 cm). 100 μl of the composition is then administered evenly between sites A and B as the needle is withdrawn. The needle is then inserted intradermally at site C, advanced in the direction of site B, and the dosing process repeated. When administration to one margin of the wound has been completed, administration may then be repeated on the other margin.

### EXPERIMENTAL RESULTS

FIG. 1

FIG. 1 illustrates data from a clinical trial conducted by the inventors to generate a dose response curve indicative of the anti-scarring effect achieved using various different doses of TGF-β3 administered in a single incidence of treatment. Either TGF-β3 or placebo were administered as a single intradermal injection to a 1 centimetre experimental wound. The figure displays the treatment effect with TGFβ3 as least square means and 95% confidence intervals from an analysis of variance (ANOVA) with site as a factor. To test the treatment effect, ToScar of the TGFβ3 scar was subtracted from the anatomically matched Placebo ToScar on the other arm on each subject. ToScar was calculated as the sum of VAS scores (mm) from week 6 and months 3, 4, 5, 6 and 7. The scars were scored by an independent lay panel at 6 time points after dosing (week 6, months 3-7) using a 100 mm VAS line.

FIG. 1 illustrates that scarring is effectively inhibited by a single application of 50 ng, 200 ng and 500 ng/100 μl TGFβ3 per cm of wound margin. The level of improvement displays a typical bell-shaped dose-response curve with maximum improvement (average >50 mm scar improvement in TGFβ3 treated wounds) observed at the 200 ng/100 μl dose, with a reduction in drug efficacy towards the top of the dose range i.e. 500 ng/100 μl per cm of wound margin

FIG. 2

FIG. 2 illustrates data from a clinical trial conducted by the inventors. In this study TGFβ3 and Placebo were each administered in two separate incidences of treatment (by means of two intradermal injections). However, unlike the methods of the present invention, the first incidence of treatment took place immediately prior to wounding but the second incidence of treatment occurred immediately after wound closure, i.e., both doses being administered within approximately 1 hour of one another (the first ten to thirty minutes prior to wounding, and the second ten to thirty minutes post-wounding). The figure displays the treatment effect with TGFβ3 as least square means and 95% confidence intervals from an analysis of variance (ANOVA) with site as a factor. To test the treatment effect, ToScar of the TGFP3 scar was subtracted from the anatomically matched Placebo ToScar on the other arm on each subject. ToScar was calculated as the sum of VAS scores (mm) from week 6 and months 3, 4, 5, 6 and 7. The scars were scored by an independent lay panel at 6 time points after dosing (week 6, months 3-7) using a 100 mm VAS line.

FIG. 2 illustrates that scarring is effectively inhibited by two applications of 5 ng, 50 ng, 200 ng and 500 ng/100 μl TGFβ3 per cm of wound margin, prior to and immediately after wound closure (i.e. both doses within approximately 1 hour). The level of improvement, displays a typical bell-shaped dose-response curve with maximum improvement (average >40 mm scar improvement in TGFβ3 treated wounds) observed at the 200 ng/100 μl dose, with a reduction in drug efficacy towards the top of the dose range i.e. 500 ng/100 μl per cm of wound margin. The degree of improvement and dose-response curve with TGFβ3 treatment given twice (within approximately 1 hour) is comparable to that for TGFβ3 given once (see FIG. 1), though over all the degree to which scarring is inhibited is slightly less than for the single administration regime. This illustrates that repeated administration of TGF-β3 (other than in the methods described in the present invention) does not lead to a greater inhibition of scarring, and if anything may somewhat diminish the anti-scarring efficacy of this compound.

FIG. 3

FIG. 3 shows data generated using the methods of the invention, where TGFβ3 and Placebo were administered in two incidences of treatment (each by intradermal injection), the first prior to wounding and the second approximately 24 hours after wounding. The figure displays the treatment effect with TGFβ3 as least square means and 95% confidence intervals from an analysis of variance (ANOVA) with site as a factor. To test the treatment effect, ToScar of the TGFβ3 scar was subtracted from the anatomically matched Placebo ToScar on the other arm on each subject. ToScar was calculated as the sum of VAS scores (mm) from week 6 and months 3, 4, 5, 6 and 7. The scars were scored by an independent lay panel at 6 time points after dosing (week 6, months 3-7) using a 100 mm VAS line.

FIG. 3 illustrates that scarring is effectively inhibited by two applications of 5 ng, 50 ng, 200 ng and 500 ng/100 μl TGFβ3 per cm of wound margin, prior to and at approximately 24 hours post-wounding. Of these experimental methods of treatment, the method in accordance with the present invention (i.e. the method in which 500 ng TGF-β3 is administered in incidences separated by 24 hours) is notably more effective than the others.

In light of previous data given in FIGS. 1 and 2 the level of improvement surprisingly does not display a typical bell-shaped dose-response curve. Furthermore, the finding that the greatest degree of improvement is achieved with TGFβ3 treatment given twice at the 500 ng/100 μl dose (prior to wounding and approximately 24 hours post-wounding) is surprising (yielding an average >60 mm scar improvement in wounds treated with TGFβ3 in this manner).

FIG. 4

FIG. 4 shows representative macroscopic images from three subjects illustrating the different extents to which scarring may be inhibited using different TGFβ3 treatment regimes. The macroscopic images are from within subject scars produced on healing of placebo treated and TGFβ3 treated wounds (dosed twice with 50 ng, 200 ng and 500 ng/100 μl TGFβ3 per cm of wound margin approximately 24 hours apart) in a clinical trial conducted by the inventors. The same amount of TGF-β3 was administered in each incidence of treatment, and the amounts used are shown in the captions (50 ng/100 μl TGFβ3 per cm of wound margin shown top left, with placebo from the same subject top right; 200 ng/100 μl TGFβ3 per cm of wound margin shown middle left, with placebo from the same subject middle right; and 500 ng/100 μl TGFβ3 per cm of wound margin shown bottom left, with placebo from the same subject bottom right).

The wound treated with the method of the invention (bottom left) can be seen to benefit from the greatest inhibition of scarring achieved.

FIG. 5

FIG. 5 shows 3-dimensional simulations and scar measurements obtained from profilometry analysis of silicone moulds from scars produced on healing of placebo treated and TGFβ3 treated wounds (dosed twice with 100 μl of 50 ng/100 μl TGFβ3 or 100 μl placebo per cm of wound margin approximately 24 hours apart) in a clinical trial conducted by the inventors. Note that this is not a method of treatment in accordance with the invention, but (along with FIG. 6) serves to provide comparative data illustrating the surprising effectiveness of a method of treatment in accordance with the invention (the results of which are set out in FIG. 7).

The top panel shows the original 3-dimensional simulations and for clarity the bottom panel illustrates the boundaries of the scars demarcated by white arrowheads, with the remaining area of the image being normal skin surrounding the scar. A range of quantitative parameters for each scar were analysed by profilometry and demonstrated a 30.21% reduction in scar surface area with TGFβ3 treatment compared to placebo (TGFβ3 treated wound scar surface area=12.823 mm²; placebo treated wound scar surface area=18.375 mm²).

FIG. 6

FIG. 6 shows 3-dimensional simulations and scar measurements obtained from profilometry analysis of silicone moulds from scars produced on healing of placebo treated and TGFβ3 treated wounds (dosed twice with 100 μl of 200 ng/100 μl TGFβ3 or 100 μl placebo per cm of wound margin approximately 24 hours apart) in a clinical trial conducted by the inventors. As with the results shown in FIG. 6, this does not constitute a method of treatment in accordance with the invention, but instead serves to provide comparative data illustrating the surprising effectiveness of a method of treatment in accordance with the invention (the results of which are set out in FIG. 7).

The top panel shows the original 3-dimensional simulations and for clarity the bottom panel illustrates the boundaries of the scars demarcated by white arrowheads, with the remaining area of the image being normal skin surrounding the scar. A range of quantitative parameters for each scar were analysed by profilometry and demonstrated a 75.19% reduction in scar surface area with TGFβ3 treatment compared to placebo (TGFβ3 treated wound scar surface area=3.532 mm²; placebo treated wound scar surface area=14.239 mm²). Profilometry analysis also demonstrated a reduction in scar raised volume with TGFβ3 treatment of 73.33% compared to placebo treatment (TGFβ3 treated wound scar raised volume=0.0008 mm³; placebo treated wound scar raised volume=0.003 mm³).

FIG. 7

FIG. 7 shows 3-dimensional simulations and scar measurements obtained from profilometry analysis of silicone moulds from scars produced on healing of placebo treated and TGFβ3 treated wounds (dosed twice with 100 μl of 500 ng/100 μl TGFβ3 or 100 μl placebo per cm of wound margin approximately 24 hours apart) in a clinical trial undertaken to illustrate the surprising effectiveness of the methods of the invention.

The top panel shows the original 3-dimensional simulations and for clarity the bottom panel illustrates the boundaries of the scars demarcated by white arrowheads, with the remaining area of the image being normal skin surrounding the scar. As can be seen, there was no detectable scar following TGFβ3 treatment, i.e. profilometry techniques could not distinguish the scar produced on healing of a wound treated with the methods of the invention from surrounding normal skin that had not been wounded. Thus the quantitative parameters analysed by profilometry demonstrated a 100% reduction in scar surface area and scar raised volume with TGFβ3 treatment in accordance with the present invention as compared to placebo since there was no scar detectable following TGFβ3 treatment (placebo treated wound scar surface area=12.711 mm²).

The results set out in FIG. 7 clearly show the surprising efficacy of the methods of the invention for the inhibition of scarring formed on healing of a wound, as compared to other (therapeutically effective) treatments using TGF-β3.

FIG. 8

FIG. 8 illustrates data from a clinical trial conducted by the inventors in which either TGF-β3 or placebo were administered in two incidents of treatment (each comprising administration of the test substance by intradermal injection), the first incidence occurring prior to wounding and the second immediately after wound closure, i.e., both doses within approximately 1 hour (10-30 mins prior to wounding and 10-30 mins post wounding). It will be recognised that the experimental methods of treatment, the results of which are shown in FIG. 8, do not represent methods of treatment in accordance with the present invention, but are instead alternative (therapeutically effective) methods of treatment that illustrate the surprising efficacy of the methods of the invention.

FIG. 8 displays the treatment effect with TGF-β3 (here labelled “Juvista”) and placebo as mean visual analogue scale (VAS) scores (mm). The scars were scored by an independent lay panel at 6 time points after dosing (week 6 and months 3-7) using a 100 mm VAS line.

FIG. 8 illustrates that scarring is inhibited by two applications of 100 μl of 5 ng, 50 ng, 200 ng and 500 ng/100 μl TGF-β3 per cm of wound margin administered prior to and immediately after wound closure (i.e. both doses within approximately 1 hour). The level of improvement is dose responsive and typically is first evident at early time points (week 6 onwards) and is maintained throughout the assessment period (i.e., up to 7 months in this study).

indicates significant difference (p<0.05) between TGF-β3 and placebo treatment

FIG. 9

FIG. 9 illustrates data from a clinical trial conducted by the inventors comparing methods of treatment in accordance with the invention with other therapeutically effective anti-scarring treatments using TGF-β3. The results illustrate the surprising efficacy of methods of the invention as compared to other treatments.

TGFβ3 and Placebo were administered by means of intradermal injection in two incidences of treatment, the first prior to wounding and the second approximately 24 hours later. The figure displays the treatment effect with TGFβ3 (once more labelled “Juvista”) and placebo as mean visual analogue scale (VAS) scores (mm). The scars were scored by an independent lay panel at 6 time points after dosing (week 6, months 3-7) using a 100 mm VAS line.

FIG. 9 illustrates that scarring is inhibited by two applications of 100 μl of 5 ng, 50 ng, 200 ng or 500 ng/100 μl TGFβ3 per cm of wound margin administered prior to wounding and at approximately 24 hour post-wounding. The level of improvement is dose responsive and typically is first evident at early time points (week 6 onwards) and is maintained throughout the assessment period (i.e., up to 7 months in this study). Surprisingly the magnitude of effect is much larger than expected from previous data. It can be seen that the method of the invention (in which 500 ng of TGF-β3 is provided per centimetre of the body site treated in each incidence of treatment) is surprisingly more effective than the other methods of treatment (which are themselves still therapeutically effective).

indicates significant difference (p<0.05) between TGFβ3 and Placebo treatment

FIG. 10

The surprisingly beneficial effects of the methods and medicaments of the invention are made even more apparent when the anti-scarring effect provided by these methods is compared with that achieved by experimental treatment regimes using the European Medicines Agency's preferred scarring assessment tool, the Global Scar Comparison Scale (GSCS).

The GSCS is described elsewhere in the specification, and comprises a “double VAS” which is able to yield greater sensitivity in the assessment of anti-scarring effects than are traditional “single VAS” approaches.

FIG. 10 shows the results of a comparison of scarring occurring after healing of wounds treated with either a method of treatment in accordance with the present invention, or one of three experimental control treatments. Surgical incisions in patients were treated with two incidences of treatment administered approximately twenty-four hours apart from one another. In each incidence of treatment TGF-β3 was administered by intradermal injection at an amount of 5 ng, 50 ng of 200 ng per treated centimetre (in the experimental treatments) or 500 ng per centimetre in the treatment in accordance with the present invention. Scarring was assessed 12 months after surgery, using the GSCS. The higher the value achieved on the Y axis, the greater the ability of the given treatment to inhibit scarring.

The results shown in A were those produced by investigating surgeons when comparing treated and control treated scars on the patient. These illustrate that the inhibition of scarring observed on healing of wounds treated with the methods of the invention is much greater than that observed on healing of control treated wounds using alternative treatment regimes.

Even more impressive are the results achieved when the scar-inhibitory capacity of the various treatments is compared by an independent clinical assessment panel using the GSCS (the combination of assessors and assessment tool agreed by the EMEA for clinical assessment of scar inhibition endpoints). These are shown in B, and clearly illustrate that the scar inhibitory effect achieved by treatment with the methods of the invention is almost twice that achieved when wounds are experimentally treated with 200 ng of TGF-β3 per centimetre (the dose suggested in the prior art as being most effective).

These results clearly illustrate that treatment of human wounds with the medicaments or methods of the invention is able to lead to an inhibition of scarring of 35% or more when assessed using a regulatory authority-validated clinical assessment tool.

Sequence Information

TGF-β 3
(Sequence ID No. 1)
ALDTNYCFRNLEENCCVRPLYIDFRQDLGWKWVHEPKGYYANFCSGPCPY
LRSADTTHSTVLGLYNTLNPEASASPCCVPQDLEPLTILYYVGRTPKVEQ
LSNMVVKSCKCS
DNA encoding wild-type human TGF-β3
Sequence ID No. 2
GCT TTG GAC ACC AAT TAC TGC TTC CGC AAC TTG GAG
GAG AAC TGC TGT GTG CGC CCC CTC TAC ATT GAC TTC
CGA CAG GAT CTG GGC TGG AAG TGG GTC CAT GAA CCT
AAG GGC TAC TAT GCC AAC TTC TGC TCA GGC CCT TGC
CCA TAC CTC CGC AGT GCA GAC ACA ACC CAC AGC ACG
GTG CTG GGA CTG TAC AAC ACT CTG AAC CCT GAA GCA
TCT GCC TCG CCT TGC TGC GTG CCC CAG GAC CTG GAG
CCC CTG ACC ATC CTG TAC TAT GTT GGG AGG ACC CCC
AAA GTG GAG CAG CTC TCC AAC ATG GTG GTG AAG TCT
TGT AAA TGT AGC

1. A method of inhibiting scarring formed on healing of a wound of a human, the method comprising treating a body site in which scarring is to be inhibited:
in a first incidence of treatment providing to each centimetre of wound margin, or each centimetre of a site at which a wound is to be formed an amount of between approximately 350 ng and 1000 ng of TGF-β3; and
in a second incidence of treatment, occurring after a wound is formed and between 8 and 48 hours after the first incidence of treatment, providing to said wound an amount of between approximately 350 ng and 1000 ng of TGF-β3 per centimetre of wound margin in which scarring is to be inhibited.

2. The method according to claim 1, wherein the TGF-β3 is provided by means of a local injection.

3. The method according to claim 2, wherein the first incidence of treatment is provided at a site where a wound is to be formed and the local injection is to be administered substantially along the midline of the wound to be formed.

4. The method according to claim 2, wherein the first incident of treatment is provided to a site at which a wound is to be formed and wherein a local injection is administered to each of the margins of the wound to be formed.

5. The method according to claim 2, wherein the first and or second incidence of treatment is provided to a wound margin and the local injection is administered at a location within half a centimetre of the wound margin

6. The method according to claim 1, wherein the first and/or second incidence of treatment comprises providing the TGF-β3 to a region extending at least half a centimetre beyond each end of the wound.

7. A method of inhibiting scarring formed on healing of a wound of a human, the method comprising treating a body site in which scarring is to be inhibited:
in a first incidence of treatment providing to each centimetre of a site where a wound is to be formed an amount of between approximately 350 ng and 1000 ng of TGF-β3; and
in a second incidence of treatment, occurring between 8 and 48 hours after the first incidence of treatment, providing to said wound an amount of between approximately 350 ng and 1000 ng of TGF-β3 per centimetre of wound margin in which scarring is to be inhibited.

8. A method of inhibiting scarring formed on healing of a wound of a human, the method comprising treating a body site in which scarring is to be inhibited:
in a first incidence of treatment providing to each centimetre of wound margin, or each centimetre of future wound margin, an amount of between approximately 350 ng and 1000 ng of TGF-β3; and
in a second incidence of treatment, occurring between 8 and 48 hours after the first incidence of treatment, providing to said wound an amount of between approximately 350 ng and 1000 ng of TGF-β3 per centimetre of wound margin in which scarring is to be inhibited.

9. A method according to claim 8, wherein the amount of TGF-β3 provided is substantially the same in each incidence of treatment.

10. A method according to claim 8, wherein the amount of TGF-β3 provided per centimetre of wound margin, or potential wound margin, in each incidence of treatment is approximately 500 ng.

11. A method according to claim 8, further comprising a third or further incidence of treatment.

12. A method according to claim 8, wherein the incidences of treatment are separated by approximately 24 hours.

13. A method according to claim 8, wherein the wound is a skin wound.

14. The method according to claim 8, where the wound is a wound of the circulatory system

15. A method according to claim 8, wherein the wound is a result of surgery.

16. A method according to claim 8, wherein the TGF-β3 is provided by local injection administered to the body site.

17. A method according to claim 8, wherein the TGF-β3 is administered in a pharmaceutically acceptable solution, approximately 100 μl of which is administered per centimetre of body site treated.

18. A method according to claim 8, wherein the first incidence of treatment occurs prior to wounding.

19. A method according to claim 18, wherein the first incidence of treatment occurs up to an hour prior to wounding.

20. A method according to claim 8, wherein the first incidence of treatment occurs after wounding.

21. A method according to claim 20, wherein the first incidence of treatment occurs up to two hours after wounding.

22. A method according to claim 8, wherein the first incidence of treatment occurs after wound closure.

23. A method according to claim 22, wherein the first incidence of treatment occurs up to two hours after wound closure.

24. A method of selecting an appropriate treatment regime for inhibiting scarring associated with the healing of a wound of a human, the method comprising:
determining whether an individual in need of such inhibition of scarring will be able to complete a second incidence of treatment occurring between 8 and 48 hours after a first incidence of treatment;
if the individual will be able to complete a second incidence of treatment occurring between 8 and 48 hours after a first incidence of treatment, selecting a treatment regime comprising treating a body site in which scarring is to be inhibited such that:
in a first incidence of treatment providing to each centimetre of wound margin, or each centimetre of a site at which a wound is to be formed, in which scarring is to be inhibited an amount of between approximately 350 ng and 1000 ng of TGF-β3; and
in a second incidence of treatment, occurring between 8 and 48 hours after the first incidence of treatment, providing to said wound an amount of between approximately 350 ng and 1000 ng of TGF-β3 per centimetre of wound margin in which scarring is to be inhibited; or
if the individual will not be able to complete a second incidence of treatment occurring between 8 and 48 hours after a first incidence of treatment, selecting a treatment regime comprising:
in a single incidence of treatment providing to each centimetre of wound margin, or each centimetre of a site at which a wound is to be formed, in which scarring is to be inhibited an amount of between approximately 150 ng and 349 ng TGF-β3.

25. TGF-β3 for use as a medicament in treating a human wound or site where a human wound is to be formed to inhibit scarring, wherein in a first incidence of treatment the medicament is provided such that between approximately 350 ng and 1000 ng of TGF-β3 is provided to each centimetre of a wound margin or each centimetre of a site at which a wound is to be formed and wherein in a subsequent incidence of treatment the medicament is provided such that between approximately 350 ng and 1000 ng of TGF-β3 is provided to each centimetre of a wound margin between 8 hours and 48 hours after the previous incidence of treatment.

26. TGF-β3 used according to claim 25, wherein the medicament is an injectable medicament.

27. TGF-β3 used according to claim 26, wherein the medicament is for intradermal injection.

28. TGF-β3 used according to any claim 25, wherein the medicament is formulated such that the requisite amount of TGF-β3 is provided in a 100 μl volume of the medicament.

29. A kit for use in the inhibition of scarring associated with healing of a human wound, the kit comprising at least first and second vials comprising TGF-β3 for administration to a wound, or a site where a wound is to be formed, at times between 8 and 48 hours apart from one another.

30. A kit for use in the inhibition of scarring associated with healing of a human wound, the kit comprising:
a first amount of a composition containing TGF-β3, this first amount being for administration to a wound, or a site where a wound is to be formed, in a first incidence of treatment;
a second amount of a composition containing TGF-β3, this second amount being for administration to a wound in a second incidence of treatment;
instructions regarding administration of the first and second amounts of the composition at times between 8 and 48 hours apart from one another;
wherein the composition is formulated to provide an amount of between approximately 350 ng and 1000 ng TGF-β3 to each centimetre of a tissue or organ to which it is administered.

31. A kit according to claim 30, wherein the composition contains TGF-β3 at a concentration of about 500 ng/100 μl.

32. A method according to claim 1, wherein the amount of TGF-β3 provided is substantially the same in each incidence of treatment.

33. A method according to claim 1, wherein the amount of TGF-β3 provided per centimetre of wound margin, or potential wound margin, in each incidence of treatment is approximately 500 ng.

34. A method according to claim 1, further comprising a third or further incidence of treatment.

35. A method according to claim 1, wherein the incidences of treatment are separated by approximately 24 hours.

36. A method according to claim 1, wherein the wound is a skin wound.

37. The method according to claim 1, where the wound is a wound of the circulatory system

38. A method according to claim 1, wherein the wound is a result of surgery.

39. A method according to claim 1, wherein the TGF-β3 is provided by local injection administered to the body site.

40. A method according to claim 1, wherein the TGF-β3 is administered in a pharmaceutically acceptable solution, approximately 100 μl of which is administered per centimetre of body site treated.

41. A method according to claim 7, wherein the amount of TGF-β3 provided is substantially the same in each incidence of treatment.

42. A method according to claim 7, wherein the amount of TGF-β3 provided per centimetre of wound margin, or potential wound margin, in each incidence of treatment is approximately 500 ng.

43. A method according to claim 7, further comprising a third or further incidence of treatment.

44. A method according to claim 7, wherein the incidences of treatment are separated by approximately 24 hours.

45. A method according to claim 7, wherein the wound is a skin wound.

46. The method according to claim 7, where the wound is a wound of the circulatory system

47. A method according to claim 7, wherein the wound is a result of surgery.

48. A method according to claim 7, wherein the TGF-β3 is provided by local injection administered to the body site.

49. A method according to claim 7, wherein the TGF-β3 is administered in a pharmaceutically acceptable solution, approximately 100 μl of which is administered per centimetre of body site treated.

50. A method according to claim 7, wherein the first incidence of treatment occurs prior to wounding.

51. A method according to claim 50, wherein the first incidence of treatment occurs up to an hour prior to wounding.

52. A method according to claim 7, wherein the first incidence of treatment occurs after wounding.

53. A method according to claim 52, wherein the first incidence of treatment occurs up to two hours after wounding.

54. A method according to claim 7, wherein the first incidence of treatment occurs after wound closure.

55. A method according to claim 54, wherein the first incidence of treatment occurs up to two hours after wound closure.
